(19) United States
(12) Patent Application Publication
(10) Pub. No.: US 2007/0156229 A1
(43) Pub. Date: Jul. 5, 2007

(54) "D"-shape stent for treatment of abdominal aortic aneurysm

(76) Inventor: Jin S. Park, Parsippany, NJ (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 11/323,013

(22) Filed: Dec. 30, 2005

(51) Int. Cl. A61F 2/06

(52) U.S. Cl. 623/1.15

(57) ABSTRACT

A bifurcated prosthetic stent graft has two stent grafts with co-located respective first ends, and disparately located respective second ends. Each stent grafts includes a first stent segment at the first end of the stent graft having a semicircular portion and a diameter portion connecting the ends of the semicircular portion and held in tension by the ends of the semicircular portion, defining a substantially D-shape. Compression and heat applied to the diameter portion during manufacture of the stent results in the diameter portion being in tension when the stent is deployed. One or more transition segments transitions the stent graft between a substantially D-shape and a substantially circular shape. A vascular graft encloses the first stent segment and one or more transition stent segments, the vascular graft providing a fluid flow lumen from the first end of the stent graft to the second end.

### BACKGROUND OF THE INVENTION

1. Field of Invention

The invention relates to the field of medical devices, and more specifically to a prosthesis for the treatment of vascular disease, particularly abdominal aortic aneurysm.

2. Description of Related Art

Vascular disease is a leading cause of premature mortality in developed nations, often presenting as a vascular aneurysm. A vascular aneurysm is a localized dilation of a vessel wall, due to thinning or weakness of the wall structure, or separation between layers of the vessel wall. If untreated, the aneurysm may burst and hemorrhage uncontrollably. Aneurysms are particularly dangerous and prevalent in the aorta, because the aorta supplies blood to all other areas of the body, and because the aorta is subject to particularly high pressures and stresses accordingly. Rupture of an aortic aneurysm is the 15^{th} leading cause of death the United States, afflicting 5% of older men.

Aortic aneurysms are described by their position. They are either thoracic, generally between the aortic arch and the junction of the left and right renal arteries, or abdominal, between the junction of the renal arteries and the branch of the iliac arteries.

It is known to treat aortic aneurysms surgically where blood pressure control medication is unsuccessful at arresting growth of the aneurysm. Surgery often involves the insertion of a vascular stent graft to exclude the aneurysm and carry blood past the dilated portion of the vessel, relieving the pressure on the aneurysm. Designing a viable stent graft for the treatment of abdominal aortic aneurysm (AAA) is particularly challenging, in part because the graft must branch to follow the shape of the abdominal aorta to carry blood into the separate iliac arteries without obstruction. Moreover, it would be advantageous to design a stent graft that is collapsible to facilitate percutaneous insertion by minimally invasive surgical techniques.

### BRIEF SUMMARY OF THE INVENTION

Provided according to the present invention is a method of forming a prosthetic stent, and a stent formed according to the method. The method includes providing a stent having a semicircular portion and a diameter portion connecting the ends of the semicircular portion. The stent may be cut, for example, laser cut, from a unitary cylinder of material, preferably a shape memory material and more preferably Nitinol or a Nitinol alloy, and may be shape-set in the shape of a semicircular portion and a diameter portion connecting the ends of the semicircular portion. A compressive force is applied to the diameter portion, which is then heated, preferably to between about 375 and about 650 degrees C., and more preferably between about 400 degrees C. and about 600 degrees C., while under the compressive force. Heat may be applied by one or more of resistance heating, air heating, laser heating, induction heating, and hot die application. Upon releasing the compressive force, the ends of the semicircular portion hold the diameter portion in tension.

Also provided according to the present invention is a prosthetic stent having a semicircular portion, and a diameter portion connecting the ends of the semicircular portion, the diameter portion being held in tension by the ends of the semicircular portion. The prosthetic stent may include a vascular graft surrounding the stent. The prosthetic stent preferably comprises a shape memory material, more preferably Nitinol or a Nitinol alloy. The prosthetic stent may be comprised of a plurality of struts arranged in a repeating diamond pattern.

Also provided according to the present invention is a bifurcated prosthetic stent graft for a bifurcated lumen, the bifurcated prosthetic stent graft having two stent grafts with co-located respective first ends, and disparately located respective second ends. Each of the two stent grafts includes a first stent segment at the first end of the stent graft having a semicircular portion and a diameter portion connecting the ends of the semicircular portion, the diameter portion being held in tension by the ends of the semicircular portion such that the first stent segment defines a substantially D-shape. One or more transition segments transitions the stent graft between a substantially D-shape on one end and a substantially circular shape on the opposite end. The opposite end of the transition segment is in communication with the second end of the stent graft. A vascular graft encloses the first stent segment and one or more transition stent segments, the vascular graft providing a fluid flow lumen from a first end of the stent graft to the second end of the stent graft.

### BRIEF DESCRIPTION OF THE DRAWINGS

These and other features, benefits, and advantages of the present invention will be made apparent with reference to the following detailed description, appended claims, and accompanying figures, wherein like reference numerals refer to like structures across the several views, and wherein:

FIGS. 1A, 1B, 1C and 1D illustrate a single D-shape stent according to an embodiment of the present invention in perspective view, side elevation view, plan view, and front elevation view, respectively;

FIGS. 2A, 2B, 2C and 2D illustrate a double D-shape stent assembly according to an embodiment of the present invention in perspective view, side elevation view, plan view, and front elevation view, respectively;

FIG. 3 illustrates a stent graft for the treatment of abdominal aortic aneurysm according to the present invention;

FIGS. 4A and 4B illustrate the effect of radial compression on a D-shape stent according to less preferred embodiments of a D-shape stent; and

FIG. 5 illustrates the effect of radial compression on a D-shape stent according to a preferred embodiment of the present invention.

### DETAILED DESCRIPTION OF THE INVENTION

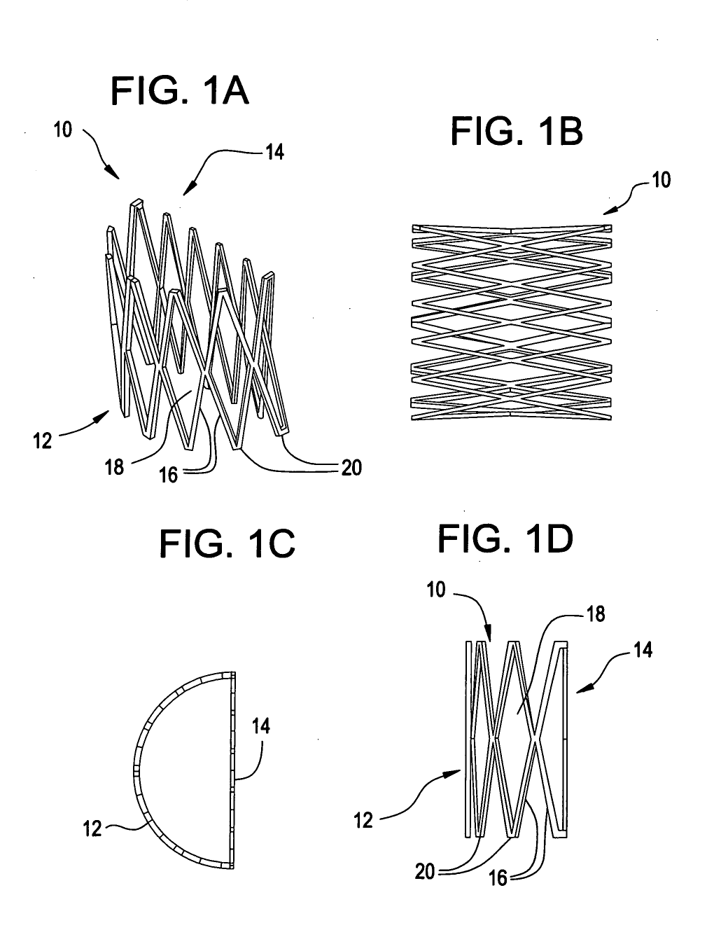

Referring now to FIGS. 1A-1D, illustrated is a single D-shape stent, generally 10, according to the present invention. The stent 10 has a semicircular portion 12, and a diameter portion 14 connecting the ends of the semicircular portion 12. The stent 10 is formed of plural struts 16. The struts 16 are arranged in a diamond pattern, where four adjacent struts 16 define the borders of a diamond 18, with this pattern repeating in the stent 10. However, other strut patterns are known in the art and may be substituted without departing from the present invention.

D-shape stent 10 preferably comprises a shape memory material, a group that includes, but is not limited to, Nitinol or a Nitinol alloy, examples of the latter including Nitinol Niobium (NiTi—Nb), Nitinol Platinum (NiTi—Pt), or Nitinol Tantalum (NiTi—Ta). D-shape stent 10 can be formed by cutting the stent from a cylindrical tube of Nitinol or a Nitinol alloy, for example by a laser-cutting technique as is known in the art, and shape-setting the stent 10 into D-shape as shown.

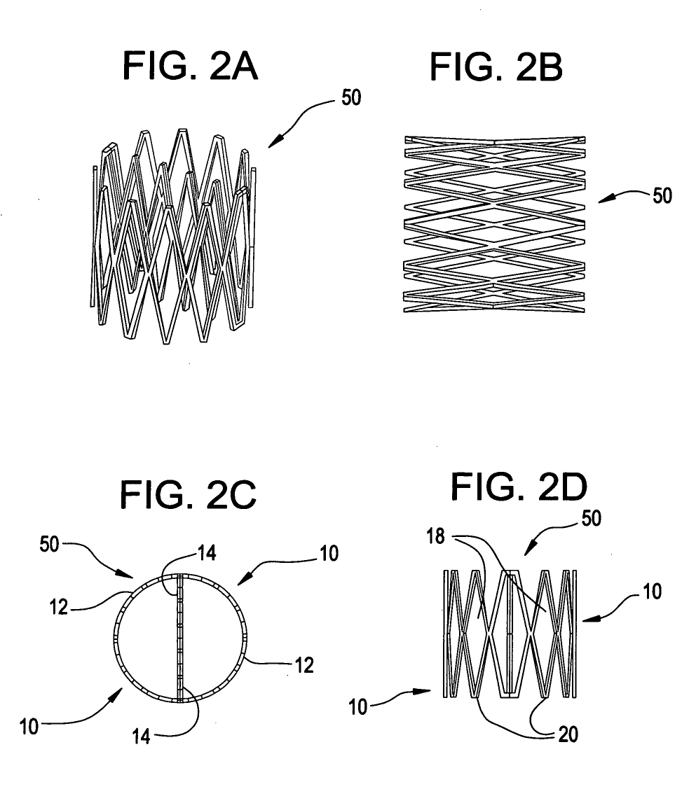

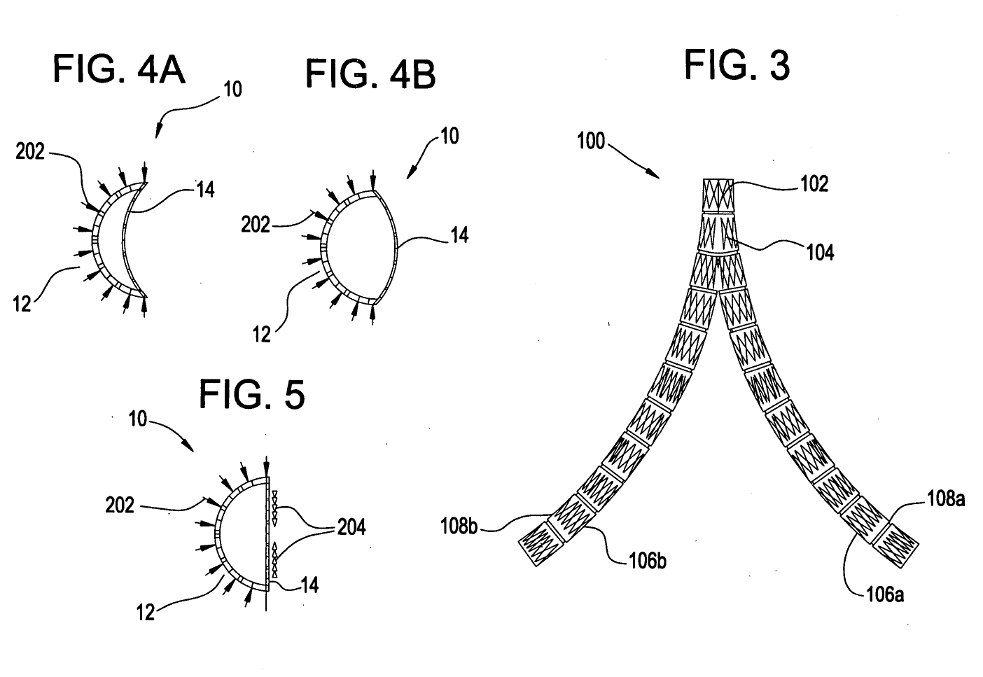

Referring now to FIGS. 2A-2D, illustrated is a double D-stent assembly, generally 50, according to the present invention. The double D-stent assembly 50 is comprised of two individual D-stents 10, oriented with their respective diameter portions 14 adjacent and touching. One or more barbs at or near the respective diameter portions 14 of each D-Shape stent 10 and extending outward from the diameter portion 14 may be provided to interlock with the stent structure or associated graft of the other and resist migration. The two D-stents 10 can be located in the abdominal aorta of a patient to form the structure of a circular inlet to a bifurcated AAA stent graft 100 (See FIG. 3). Accordingly, the two branches 106a, 106b, of the AAA stent graft 100 can be compressed to a low profile, and introduced to the aorta percutaneously, with all the associated advantages of minimally invasive surgical methods and techniques.

The diamonds 18 of each D-shape stent 10 have points 20 at the bottom where additional stent segments can be attached or at least generally aligned. The additional segments will either transition from the substantially D-shape of the stent 10 to a circular shape, or be circular shaped.

Referring now to FIG. 3, a bifurcated AAA stent graft, generally 100, is illustrated. Two D-stents 10 positioned as in assembly 50 form the first segment 102 of the stent graft 100. Thereafter, transition segment 104 transitions between the substantially D-shape of stents 10 and the circular shape of branch 106a, 106b. Each branch 106a, 106b, includes a vascular graft 108a, 108b, carried by and surrounding D-shape stents 10, and additional transition or circular stent segments, to provide a fluid flow path through the respective branch 106a, 106b.

Referring now to FIGS. 4A and 4B, when inserted in the aorta, the semicircular portion 12 will be subjected to radially inward compressive stress by the vessel wall, represented by arrows 202. Absent measures such as those according to the present invention, this compression could induce a bowing or buckling of the diameter portion 14, as the compressive force reduces the distance between the ends of the semicircular portion 12, as illustrated in FIGS. 4A, 4B.

According to the present invention, the diameter portion 14 is placed under tension in the deployed shape. This is accomplished, for example, by applying a compression force to the diameter portion 14 during manufacture of the D-shape stent 10. While under compression, a localized heat treatment is applied only to only the diameter portion 14, while avoiding any heating of the semicircular portion 12. Contemplated methods of heating include, but are not limited to, hot die application, resistance heating, induction heating, laser heating, or application of heated air to the diameter portion 14. Where D-shape stent 10 is made of a Nitinol or a Nitinol alloy material, a preferred range of heating is between about 375-650 degrees C., and more preferably between about 400-600 degrees C. The heating alters the molecules of the diameter portion 14 to relive the compressive stress resulting from the applied compression force.

Moreover, the heat application is preferably localized or isolated to only the diameter portion 14. Towards this end, a heat sink can be used adjacent or near the semicircular portion 12 to direct heat away from or draw heat from the semicircular portion 12.

Following the heat treatment, having removed the influence of the compressive force, the ends of semicircular portion 12 of D-shape stent 10 hold the diameter portion 14 under tension. Referring now to FIG. 5, arrows 204 illustrate the tension in diameter portion 14 of stent 10. When the semicircular portion 12 is subjected to radial compressive force, illustrated by arrows 202, the tension in diameter portion 14 is relieved, but no buckling or bowing occurs. Accordingly, a D-shape stent 10 according to the present invention has greater dimensional stability in use.

The present invention has been described herein with reference to certain exemplary or preferred embodiments. These embodiments are offered as merely illustrative, not limiting, of the scope of the present invention. Certain alterations or modifications may be apparent to those skilled in the art in light of instant disclosure without departing from the spirit or scope of the present invention, which is defined solely with reference to the following appended claims.

1. A prosthetic stent comprising:
a semicircular portion;
a diameter portion connecting the ends of the semicircular portion, the diameter portion being held in tension by the ends of the semicircular portion.

2. The prosthetic stent according to claim 1, further comprising a vascular graft surrounding the stent.

3. The prosthetic stent according to claim 1, wherein the stent comprises a shape memory material.

4. The prosthetic stent according to claim 3, the shape-memory material comprises Nitinol or a Nitinol alloy.

5. The prosthetic stent according to claim 1, further comprising a plurality of struts arranged in a repeating diamond pattern.

6. The prosthetic stent according to claim 1, further comprising one or more barbs at or near the diameter portion and extending outward from the diameter portion.

7. A prosthetic stent formed by a process comprising:
(a) providing a stent having a semicircular portion and a diameter portion connecting the ends of the semicircular portion;
(b) applying a compressive force to the diameter portion;
(c) applying heat to the diameter portion while under the compressive force; and
(d) releasing the compressive force such that the ends of the semicircular portion hold the diameter portion in tension.

8. The prosthetic stent according to claim 7, wherein the stent further comprises a vascular graft carried by the stent.

9. The prosthetic stent according to claim 7, wherein the stent comprises a shape memory material.

10. The prosthetic stent according to claim 9, the shape-memory material comprises Nitinol.

11. The prosthetic stent according to claim 9, the shape-memory material comprises a Nitinol alloy.

12. The prosthetic stent according to claim 7, further comprising a plurality of struts arranged in a repeating diamond pattern.

13. The prosthetic stent according to claim 7, wherein the step of applying heat further comprises applying heat to a temperature of between about 375 degrees C. and about 650 degrees C.

14. The prosthetic stent according to claim 7, wherein the step of applying heat further comprises applying heat to a temperature of between about 400 degrees C. and about 600 degrees C.

15. The prosthetic stent according to claim 7, wherein the step of applying heat further comprises applying heat by one or more of resistance heating, induction heating, laser heating, air heating, and hot die application.

16. The prosthetic stent according to claim 7, wherein the step of applying heat further comprises isolating the application of heat to only the diameter portion and not to the semicircular portion.

17. The prosthetic stent according to claim 16, wherein the step of applying heat further comprises providing a heat sink to direct heat away from or draw heat from the semicircular portion.

18. A method of forming a prosthetic stent comprising:
(a) providing a stent having a semicircular portion and a diameter portion connecting the ends of the semicircular portion;
(b) applying a compressive force to the diameter portion;
(c) applying heat to the diameter portion while under the compressive force; and
(d) releasing the compressive force such that the ends of the semicircular portion hold the diameter portion in tension.

19. The method according to claim 18, wherein the providing step further comprises shape-setting the stent in the shape of a semicircular portion and a diameter portion.

20. The method according to claim 18, wherein the providing step further comprises cutting the stent from a unitary cylinder of material.

21. The method according to claim 18, wherein the providing step further comprises providing the stent comprising a shape memory material.

22. The method according to claim 21, wherein the providing step further comprises providing the stent comprising Nitinol.

23. The method according to claim 22, wherein the providing step further comprises providing the stent comprising a Nitinol alloy.

24. The method according to claim 18, wherein the step of applying heat further comprises applying heat to a temperature of between about 375 degrees C. and about 650 degrees C.

25. The method according to claim 24, wherein the step of applying heat further comprises applying heat to a temperature of between about 400 degrees C. and about 600 degrees C.

26. The method according to claim 18, wherein the step of applying heat further comprises applying heat by one or more of resistance heating, induction heating, laser heating, air heating, and hot die application.

27. The method according to claim 18, wherein the step of applying heat further comprises isolating the application of heat to only the diameter portion and not to the semicircular portion.

28. The method according to claim 27, wherein the step of applying heat further comprises providing a heat sink to direct heat away from or draw heat from the semicircular portion.

29. A bifurcated prosthetic stent graft for a bifurcated lumen, the bifurcated prosthetic stent graft comprising two stent grafts having a co-located respective first ends, and disparately located respective second ends, each of the two stent grafts comprising:
a first stent segment at the first end of the stent graft, the first stent segment having a semicircular portion and a diameter portion connecting the ends of the semicircular portion, the diameter portion being held in tension by the ends of the semicircular portion such that the first stent segment defines a substantially D-shape;
one or more transition segments which transition between a substantially D-shape on one end and a substantially circular shape on the opposite end, the opposite end of the transition segment being in communication with the second end of the stent graft; and
a vascular graft enclosing the first stent segment and one or more transition stent segments, the vascular graft providing a fluid flow lumen from a first end of the stent graft to the second end of the stent graft.
